(19) United States
(12) Patent Application Publication
(10) Pub. No.: US 2026/0183348 A1
(43) Pub. Date: Jul. 2, 2026

(54) COMPOSITION FOR INCREASING MUSCLE MASS, IMPROVING MUSCULAR FUNCTIONS, OR PREVENTING OR TREATING MUSCULAR DISEASE CONTAINING PHOCAEICOLA VULGATUS

(71) Applicant: KOREA INSTITUTE OF SCIENCE AND TECHNOLOGY, Seoul (KR)

(72) Inventors: Myungsuk KIM, Seoul (KR); Choong-Gu LEE, Seoul (KR); Young Tae PARK, Seoul (KR); Hye-Young YOUN, Seoul (KR); Sun Hee PARK, Seoul (KR); Huitae MIN, Seoul (KR)

(73) Assignee: KOREA INSTITUTE OF SCIENCE AND TECHNOLOGY, Seoul (KR)

(21) Appl. No.: 19/221,044

(22) Filed: May 28, 2025

(30) Foreign Application Priority Data
Dec. 27, 2024 (KR) 10-2024-0198550

(51) Int. Cl. A61K 35/741; A61K 35/00; A61P 21/00

(52) U.S. Cl. CPC A61K 35/741; A61P 21/00; A61K 2035/115

(57) ABSTRACT

The present invention relates to a composition for increasing muscle mass, improving muscle function, or preventing or treating muscle disease, comprising Phocaeicola vulgatus or cultures thereof. In addition, the present invention relates to a method for increasing muscle mass, improving muscle function,, or preventing or treating muscular disease, comprising administering or taking a composition comprising Phocaeicola vulgatus or a culture thereof as an active ingredient to an individual in need. The Phocaeicola vulgatus of the present invention has the outstanding effect of increasing muscle strength, increasing muscle weight, and increasing muscle fiber.

### TECHNICAL FIELD

This application claims benefit of priority based on Korean Patent Application No. 10-2024-0198550 filed on Dec. 27, 2024, the disclosures of which are incorporated herein by reference in their entireties.

The present invention relates to a composition for increasing muscle mass, improving muscle function, or preventing or treating muscle disease, comprising Phocaeicola vulgatus or cultures thereof. In addition, the present invention relates to a method for increasing muscle mass, improving muscle function, or preventing or treating muscular disease, comprising administering or taking a composition comprising Phocaeicola vulgatus or a culture thereof as an active ingredient to an individual in need.

### BACKGROUND ART

Muscle can be categorized into skeletal muscle, cardiac muscle, and visceral muscle, with skeletal muscle being the most abundant tissue in the human body, making up 40-45% of body weight. Skeletal muscles attach to bones via tendons and are responsible for generating bone movement or force. A muscle is made up of many myofibers, which in turn are made up of many myofibrils, which are made up of actin and myosin. When actin and myosin overlap and move, the length of the muscle shortens or lengthens, causing contraction and relaxation of the overall muscle. An increase in the size of the myofibrils means an increase in the thickness of the myofibers, resulting in muscle gain.

Muscular diseases are characterized by a progressive progression of skeletal muscle weakness that leads to impaired walking and mobility, difficulty with activities of daily living (ADLs), and loss of independence. In addition, they can lead to cardiopulmonary dysfunction and other complications, so it is important to accurately understand the characteristics of each muscular disease and approach it accordingly. Cachexia, one of these muscle diseases, is a complex syndrome that causes continuous muscle loss due to various causes such as cancer, heart failure, chronic obstructive pulmonary disease, chronic kidney disease, and AIDS, and is characterized by significant loss of muscle mass and weight loss.

According to the World Health Organization (WHO), the number of cancer patients worldwide reached 18 million in 2018, with 9.6 million cancer deaths each year, and 1.9 million, or 20% of them, were directly caused by cancer cachexia. However, there is currently no treatment for cancer cachexia, and although many have attempted to develop treatments targeting muscle molecules, development has been halted due to side effects or ineffectiveness.

Meanwhile, south Korea entered an aging society in 2000 with the elderly population accounting for 7.2% of the total population, and is expected to enter a super-aging society (more than 20%) in 2050 (2013 statistics on the elderly, Statistics Korea). As a person's muscle mass decreases with age (by 10-15% between the ages of 50 and 70, and by more than 30% between the ages of 70 and 80), muscle strength and muscle function also decrease, a condition known as sarcopenia. Senile sarcopenia is a major cause that limits the independent living of older people by causing activity and walking difficulties. In addition, sarcopenia lowers basal metabolic rate, increases insulin resistance, promotes type 2 diabetes, and increases the risk of high blood pressure and cardiovascular disease by 3-5 times. Currently, there are no drugs approved for the treatment of sarcopenia, and drug repositioning technology that applies myostatin inhibitors or existing FDA-approved treatments for other diseases to sarcopenia is being developed.

The present inventors have been trying to develop a therapeutic agent/a composition or a treating method that can prevent, ameliorate or treat muscle diseases such as muscle atrophy or cachexia, and have experimentally confirmed that Phocaeicola vulgatus of the present invention is very useful for increasing muscle fiber(s), increasing muscle weight, and improving muscle strength, thereby completing the present invention.

### DISCLOSURE

### Technical Problem

It is an object of the present invention to provide a method for increasing muscle mass, improving muscle function, or preventing or treating muscular disease, comprising administering or taking a composition comprising Phocaeicola vulgatus or a culture thereof as an active ingredient to an individual in need.

It is also an object of the present invention to provide a pharmaceutical composition for increasing muscle mass, improving muscle function, or preventing or treating muscle disease, comprising Phocaeicola vulgatus or a culture thereof as an active ingredient.

It is also an object of the present invention to provide a quasi-drug composition for increasing muscle mass, improving muscle function, or preventing or ameliorating muscle disease, comprising Phocaeicola vulgatus or a culture thereof as an active ingredient.

It is also an object of the present invention to provide a food composition for increasing muscle mass, improving muscle function, or preventing or ameliorating muscle disease, comprising Phocaeicola vulgatus or a culture thereof as an active ingredient.

However, the technical problem to be achieved by the present invention is not limited to the problems mentioned above, and other problems not mentioned will be clearly understood by those skilled in the art from the description below.

### Technical Solution

The present invention provides a method for increasing muscle mass, improving muscle function, or preventing or treating muscular disease, comprising administering or taking a composition comprising Phocaeicola vulgatus or a culture thereof as an active ingredient to an individual in need.

The present invention also provides a pharmaceutical composition for increasing muscle mass, improving muscle function, or preventing or treating muscle disease, comprising Phocaeicola vulgatus or a culture thereof as an active ingredient.

The present invention also provides a quasi-drug composition for increasing muscle mass, improving muscle function, or preventing or ameliorating muscle disease, comprising Phocaeicola vulgatus or a culture thereof as an active ingredient.

The present invention also provides a food composition for increasing muscle mass, improving muscle function, or preventing or ameliorating muscle disease, comprising Phocaeicola vulgatus or a culture thereof as an active ingredient.

The Phocaeicola vulgatus may be the strain Phocaeicola vulgatus KCTC 25021; ATCC 8482.

The muscle disease may be one or more diseases selected from the group consisting of sarcopenia, muscular atrophy, cachexia, myasthenia, muscular dystrophy, myotonia, hypotonia, muscular weakness, muscular dystrophy, atony, amyotrophic lateral sclerosis, and inflammatory myopathy.

The compositions may enhance muscle strength.

The composition may increase muscle weight.

The muscle may comprise one or more selected from the group consisting of quadricep muscle, soleus muscle, gastrocnemius muscle and tibialis muscle.

The composition may reduce spleen weight.

The composition may increase muscle fiber(s).

### Advantageous Effects

The Phocaeicola vulgatus or a culture thereof of the present invention is significantly effective in increasing muscle strength, increasing muscle weight, and increasing muscle fiber. Therefore, it can be applied to various products, such as a pharmaceutical composition, a food composition, and a quasi-drug composition for increasing muscle mass, improving muscle function, or preventing, treating, or ameliorating muscle disease.

### DESCRIPTION OF DRAWINGS

FIG. 1 shows the results of the efficacy of Phocaeicola vulgatus in improving muscle strength (Con: control, CT26: cancer cachexia-induction group, CT26+PV: cancer cachexia-induction+Phocaeicola vulgatus-treatment group; ***, p<0.001).

FIGS. 2 to 3 show the results of the efficacy of Phocaeicola vulgatus in increasing the weight of quadricep muscle and tibialis muscle (Con: control, CT26: cancer cachexia-induction group, CT26+PV: cancer cachexia-induction+Phocaeicola vulgatus-treatment group; *, p<0.05; **, p<0.01; ***, p<0.001).

FIG. 4 shows the results of the efficacy of Phocaeicola vulgatus in reducing spleen weight (Con: control, CT26: cancer cachexia-induction group, CT26+PV: cancer cachexia-induction+Phocaeicola vulgatus-treatment group; *, p<0.05; ***, p<0.001).

FIGS. 5 to 6 show the results of the efficacy of Phocaeicola vulgatus in improving muscle fiber (Con: control, CT26: cancer cachexia-induction group, CT26+PV: cancer cachexia-induction+Phocaeicola vulgatus-treatment group; **, p<0.01; ***, p<0.001).

FIG. 7 shows the results of the efficacy of Phocaeicola vulgatus in improving muscle strength (Con: control; Panc02: cancer cachexia-induction group; Panc02+PV: cancer cachexia-induction+Phocaeicola vulgatus-treatment group; **, p<0.01; ***, p<0.001).

FIGS. 8 to 9 show the results of the efficacy of Phocaeicola vulgatus in increasing the weight of quadricep muscle and soleus muscle (Con: control, Panc02: cancer cachexia-induction, Panc02+PV: cancer cachexia-induction+Phocaeicola vulgatus-treatment group; *, p<0.05; ***, p<0.001).

FIG. 10 shows the results of the efficacy of Phocaeicola vulgatus in reducing spleen weight (Con: control, Panc02: cancer cachexia-induction group, Panc02+PV: cancer cachexia-induction+Phocaeicola vulgatus-treatment group; *, p<0.05; **, p<0.01).

FIGS. 11 and 12 show the results of the efficacy of Phocaeicola vulgatus in improving muscle fiber (Con: control, Panc02: cancer cachexia-induction group, Panc02+PV: cancer cachexia-induction+Phocaeicola vulgatus-treatment group; *, p<0.05; ***, p<0.001).

### MODE OF THE INVENTION

The inventors have been trying to develop a therapeutic agent/a composition or a treating method that can prevent, ameliorate or treat muscle diseases such as muscle atrophy or cachexia, and have experimentally confirmed that Phocaeicola vulgatus of the present invention is very useful for increasing muscle fiber(s), increasing muscle weight, and improving muscle strength, thereby completing the present invention.

Hereinafter, the present invention will be described in detail.

The present invention provides a method for increasing muscle mass, improving muscle function, or preventing or treating muscular disease, comprising administering or taking a composition comprising Phocaeicola vulgatus or a culture thereof as an active ingredient to an individual in need.

The present invention also provides a composition for increasing muscle mass, improving muscle function, or preventing, improving, or treating muscle disease, comprising Phocaeicola vulgatus or a culture thereof as an active ingredient.

The muscle disease may be one or more diseases selected from the group consisting of sarcopenia, muscular atrophy, cachexia, myasthenia, muscular dystrophy, myotonia, hypotonia, muscular weakness, muscular dystrophy, atony, amyotrophic lateral sclerosis, and inflammatory myopathy.

The Phocaeicola vulgatus may be the strain Phocaeicola vulgatus KCTC 25021 (other collection No. ATCC 8482).

The compositions may enhance muscle strength.

The composition may increase muscle weight.

The muscle may comprise one or more selected from the group consisting of quadricep muscle, soleus muscle, gastrocnemius muscle and tibialis muscle.

The composition may reduce spleen weight.

The composition may increase muscle fiber.

The composition may be a pharmaceutical composition, a quasi-drug composition, a food composition, or a health functional food composition.

As used herein, “preventing” means any act of delaying the onset of a muscular disease by administration of a composition of the invention, and “treating” and “ameliorating/improving” means any act of ameliorating or beneficially altering the symptoms of a muscular disease by administration of a composition of the invention.

As used herein, the term “comprising as an active ingredient” means comprising a sufficient amount to achieve the efficacy or activity of the present Phocaeicola vulgatus or a culture thereof.

The upper limit of the quantitative amount of Phocaeicola vulgatus or a culture thereof included in the composition of the present invention may be selected by those skilled in the art within any suitable range.

Pharmaceutical Composition for Increasing Muscle Mass, Improving Muscle Function or Preventing or Treating Muscle Disease, Comprising Phocaeicola vulgatus or Cultures Thereof as an Active Ingredient

The composition of the present invention can be formulated as a pharmaceutical composition.

When the composition of the present invention is formulated as a pharmaceutical composition, the pharmaceutical composition of the present invention may comprise a pharmaceutically acceptable carrier.

According to a preferred embodiment of the present invention, the composition of the present invention may be a pharmaceutical composition comprising (a) a pharmaceutically effective amount of the Phocaeicola vulgatus or culture thereof of the present invention as described above; and (b) a pharmaceutically acceptable carrier. As used herein, the term “pharmaceutically effective amount” means an amount sufficient to achieve the efficacy or activity of the Phocaeicola vulgatus or culture thereof described above.

Pharmaceutically acceptable carriers are those customarily used in the formulation: lactose, dextrose, sucrose, sorbitol, mannitol, starch, acacia gum, calcium phosphate, alginate, gelatin, Pharmaceutically acceptable carriers are those customarily used in the formulation: lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oils, but not limited thereto. In addition to the above ingredients, the pharmaceutical composition of the present invention may further include lubricants, wetting agents, sweeteners, flavoring agents, emulsifiers, suspending agents, preservatives, etc. Suitable pharmaceutically acceptable carriers and formulations are described in detail in Remington's Pharmaceutical Sciences (19th ed., 1995).

The pharmaceutical composition of the present invention can be administered orally or parenterally.

Suitable dosages of the pharmaceutical composition of the present invention can be prescribed in a variety of ways, depending on factors such as the method of formulation, mode of administration, patient age, weight, sex, medical condition, food, time of administration, route of administration, rate of excretion, and response sensitivity. Typical dosages of the active ingredients in the pharmaceutical composition of the present invention are in the range of 0.001-100 mg/kg/day, preferably 0.01-35 mg/kg/day in adults. The dose may be administered once daily or may be divided into several doses. However, the above dosages do not limit the scope of the present invention.

The pharmaceutical composition of the present invention may be prepared in unit dose form or by formulation with pharmaceutically acceptable carriers and/or excipients, in accordance with methods readily practiced by a person of ordinary skill in the art to which the invention belongs. The formulation may be in the form of a solution, suspension, syrup, or emulsion in an oil or aqueous medium, or in the form of an excipient, acid, powder, granule, tablet, or capsule, and may further comprise a dispersant or stabilizing agent.

Quasi-Drug Composition for Increasing Muscle Mass, Improving Muscle Function or Preventing or Ameliorating Muscle Disease, Comprising Phocaeicola vulgatus or a Culture Thereof as an Active Ingredient

The composition of the present invention can be provided as a quasi-drug composition.

The present Phocaeicola vulgatus or cultures thereof may be added as is, or may be used together with ingredients such as other quasi-drugs, and may be used appropriately according to conventional methods. The mixing amount of the active ingredient can be appropriately determined depending on the purpose of use (prevention, health, or therapeutic treatment). The quasi-drug composition may be used in the manufacture of external preparations, patches, ointments, etc., but is not limited thereto.

Food Composition for Increasing Muscle Mass, Improving Muscle Function or Preventing or Ameliorating Muscle Disease, Comprising Phocaeicola vulgatus or a Culture Thereof as an Active Ingredient

The composition of the present invention can be provided as a food composition or a health functional food composition.

When the composition comprising the Phocaeicola vulgatus or a culture thereof of the present invention as an active ingredient is manufactured as a food composition, it may include not only the Phocaeicola vulgatus or a culture thereof of the present invention, but also ingredients that are customarily added in the manufacture of food products, for example, proteins, carbohydrates, fats, nutrients, seasonings and flavorings. Examples of the carbohydrates described above are monosaccharides, e.g., glucose, fructose, and the like; disaccharides, e.g., maltose, sucrose, oligosaccharides, and the like; and polysaccharides, e.g., dextrins, cyclodextrins, and the like, and sugar alcohols, e.g., xylitol, sorbitol, erythritol, and the like. As flavoring agents, natural flavoring agents [such as thaumatin, stevia extract (e.g., rebaudioside A, glycyrrhizin, etc.)] and synthetic flavoring agents (such as saccharin, aspartame, etc.) can be used. For example, if the food composition of the present invention is formulated as a beverage, it may additionally contain citric acid, liquid dextrose, sugar, glucose, acetic acid, malic acid, fruit juice, caterpillar extract, jujube extract, licorice extract, etc. in addition to the natural product extract of the present invention.

The formulation of the food composition or health functional food composition may be in the form of an acid, granule, pill, tablet, capsule, or capsule, as well as in the form of a conventional food or beverage.

Examples of food products to which the substance may be added include, without limitation, meat, sausages, bread, chocolate, candy, snacks, confectionery, pizza, ramen, other noodles, chewing gum, dairy products including ice cream, various soups, beverages, teas, drinks, alcoholic beverages and vitamin complexes, all of which are food products in the ordinary sense of the term.

In general, when manufacturing food or beverages, the Phocaeicola vulgatus or a culture thereof of the present invention can be added in an amount of 15 parts by weight or less, preferably 10 parts by weight or less, based on 100 parts by weight of raw materials. However, in the case of long-term intake for the purpose of health and hygiene or health control, the amount may be below the above range, and the present invention may also be used in amounts above the above range, as the invention is safe in that it utilizes a natural product.

Hereinafter, the present invention will be described in more detail through examples. The purpose, features, and advantages of the present invention will be easily understood through the following examples. The present invention is not limited to the embodiments described herein and may be embodied in other forms. The embodiments introduced here are provided to enable the idea of the present invention to be sufficiently conveyed to those skilled in the art. Therefore, the present invention should not be limited by the following examples.

### [Example 1] Confirmation of Changes in Muscle Strength in Mice Induced with Cancer Cachexia Using CT26 Colon Carcinoma Cell Line

Balb/c mice (male, 6 weeks old) were obtained from Dooyeol Biotech Co., Ltd. and were divided into three experimental groups (n=10 per group) according to body weight after a 2-week adaptation period. To induce cancer cachexia in the experimental groups except the control group, 1×10⁶ cells/100 μl of colon carcinoma cell line (CT26) was injected subcutaneously into the right flank of mice and tumor formation was checked for 11 days. As a control group, PBS was injected subcutaneously into the right flank of mice to correct for this effect.

The diets, doses, and methods of administration to the experimental groups were shown in Table 1 below. The test substance was the microorganism, Phocaeicola vulgatus KCTC 25021 (other collection No. ATCC 8482) from the Korean Collection for Type Cultures (KCTC), which was prepared as a liquid suspension using PBS containing 0.2% Cysteine-Hydrochloride (Cys-hcl) and administered orally daily for 3 weeks at 1×10⁹ CFU/mouse. To compensate for the placebo effect of 0.2% Cys-hcl PBS and the weight loss caused by the stress of administration, the control group was received 0.2% Cys-hcl PBS orally daily. All mice were fed the same AlN-76 research Diets, maintained on a 12 hr: 12 hr of dark: light cycle, with water ad libitum. As shown in FIG. 1, it was confirmed that grip strength was decreased in the CT26 group (cancer cachexia-induction group) and that grip strength was increased in the CT26+PV group administered P. vulgatus (CT26+PV) compared to the CT26 group.

TABLE 1

| | | Cancer | | | |
|---|---|---|---|---|---|
| | | cachexia- | Orally | Dosage and | |
| Experimental | | inducing | administered | method of | Administration |
| group | Feed | substance | substance | administration | period |
| 1 (Con) | AIN-76 | PBS | Vehicle | 0.2% Cys-hcl | Oral |
| | diet | | | PBS/200 ul, | administration |
| | | | | once per day, | of test substance |
| | | | | orally | for 3 weeks after |
| 2 (CT26) | | CT26 cells | Vehicle | 0.2% Cys-hcl | confirming |
| | | (1 × 10⁶ cells/ | | PBS/200 ul, | tumor formation |
| | | 100 μl, 1 | | once per day, | for 11 days |
| | | subcutaneous | | orally | |
| 3 (CT26 + | | injection) | P. vulgatus | P. vulgatus 1 × | |
| PV) | | | KCTC 25021 | 10⁹ CFU/200 μl, | |
| | | | | once per day, | |
| | | | | orally | |

### [Example 2] Confirmation of Weight Gain by Muscle Type in Mice Induced with Cancer Cachexia Using CT26 Colon Carcinoma Cell Line

After a total of 3 weeks of administration of P. vulgatus in [Example 1] above, the weight of each type of muscle tissue in all-treatment groups was determined.

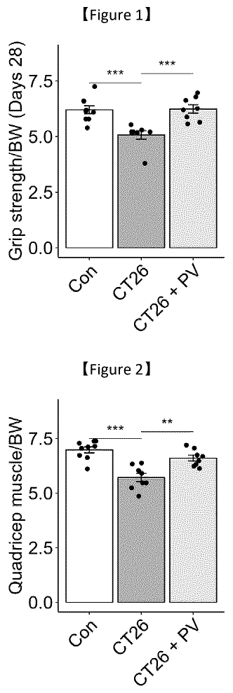

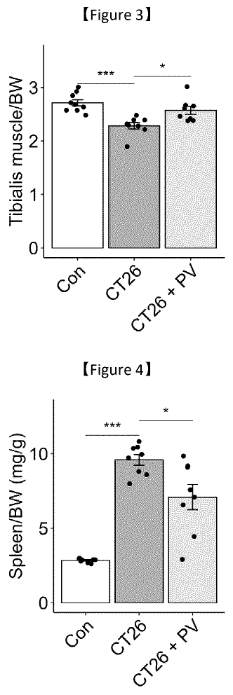

As a result, as shown in FIGS. 2 and 3, compared to the control group, it was confirmed that the CT26 group (cancer cachexia-induction group) showed a decrease in the weight of quadricep muscle and tibialis muscle, and the CT26+PV group administered P. vulgatus showed a significant increase in the weight of quadricep muscle and tibialis muscle.

### [Example 3] Confirmation of Gain and Loss of Spleen Weight in Mice Induced with Cancer Cachexia Using CT26 Colon Carcinoma Cell Line

After a total of 3 weeks of administration of P. vulgatus in [Example 1] above, the spleen weights in all-treatment groups were determined.

As a result, as shown in FIG. 4, compared to the control group, it was confirmed that the CT26 group (cancer cachexia-induction group) showed an increase in spleen weight, and the CT26+PV group administered P. vulgatus showed a significant decrease in spleen weight.

### [Example 4] Confirmation of Myofiber Improvement in Mice Induced with Cancer Cachexia Using CT26 Colon Carcinoma Cell Line

After a total of 3 weeks of administration of P. vulgatus in [Example 1] above, the tibialis muscle of in all-treatment groups was stained with H&E (hematoxylin and eosin) and histological examination was performed.

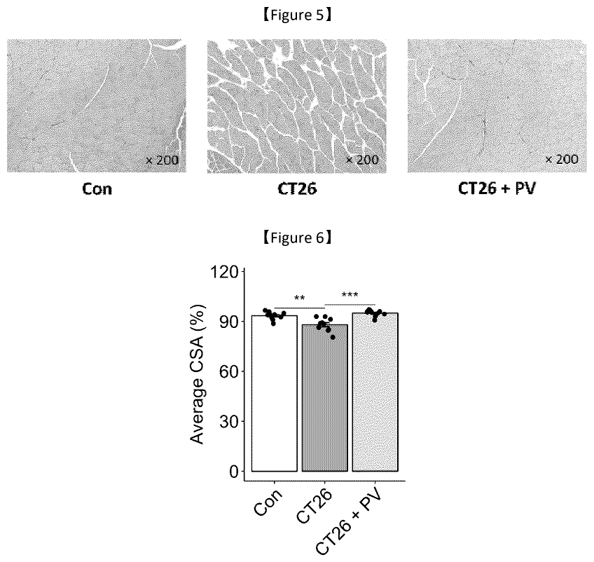

As a result, as shown in FIGS. 5 and 6, it was confirmed that the CT26+PV group administered P. vulgatus showed a significant increase in muscle fiber (cross-sectional area %) compared to the CT26 group.

### [Example 5] Confirmation of Changes in Muscle Strength in Mice Induced with Cancer Cachexia Using Panc02 Pancreatic Adenocarcinoma Cell Line

C57BL/6J (female, 6 weeks old) were obtained from Dooyeol Biotech Co., Ltd. and were divided into three experimental groups (n=10˜18 per group) according to body weight after a 2-week adaptation period. To induce cancer cachexia in the experimental groups except the control group, mice were intraperitoneally injected with 2×10⁵ cells/100 μl of pancreatic adenocarcinoma cell line (Panc02), and tumor formation was checked for 11 days. As a control group, mice were intraperitoneally injected with PBS to correct for this effect.

The diets, doses, and methods of administration to the experimental groups were shown in Table 2 below. The test substance was the microorganism, Phocaeicola vulgatus KCTC 25021 (other collection No. ATCC 8482) from the Korean Collection for Type Cultures (KCTC), which was prepared as a liquid suspension using PBS containing 0.2% Cysteine-Hydrochloride (Cys-hcl) and administered orally daily for 3 weeks at 1×10⁹ CFU/mouse. To compensate for the placebo effect of 0.2% Cys-hcl PBS and the weight loss caused by the stress of administration, the control group was received 0.2% Cys-hcl PBS orally daily. All mice were fed the same AlN-76 research Diets, maintained on a 12 hr: 12 hr of dark: light cycle, with water ad libitum. As shown in FIG. 7, it was confirmed that grip strength was decreased in the Panc02 group (cancer cachexia-induction group) and that grip strength was increased in the Panc02+PV group administered P. vulgatus compared to the Panc02 group.

TABLE 2

| | | Cancer | | | |
|---|---|---|---|---|---|
| | | cachexia- | Orally | Dosage and | |
| Experimental | | inducing | administered | method of | Administration |
| group | Feed | substance | substance | administration | period |
| 1 (Con) | AIN-76 | PBS | Vehicle | 0.2% Cys-hcl | Oral |
| | diet | | | PBS/200 μl, | administration |
| | | | | once per day, | of test substance |
| | | | | orally | for 3 weeks after |
| 2 (Panc02) | | Panc02 cells | Vehicle | 0.2% Cys-hcl | 1 week of cancer |
| | | (2 × 10⁵ cells/ | | PBS/200 μl, | cachexia- |
| | | 100 μl, 1 | | once per day, | induction |
| | | intraperitoneal | | orally | |
| 3 (Panc02 + | | injection) | P. vulgatus | P. vulgatus | |
| PV) | | | KCTC 25021 | 1 × 10⁹ | |
| | | | | CFU/200 μl, | |
| | | | | once per day, | |
| | | | | orally | |

### [Example 6] Confirmation of Weight Gain by Muscle Type in Mice Induced with Cancer Cachexia Using Panc02 Pancreatic Adenocarcinoma Cell Line

After a total of 3 weeks of administration of P. vulgatus in Example 5 above, the weight of each type of muscle tissue in all-treatment groups was determined.

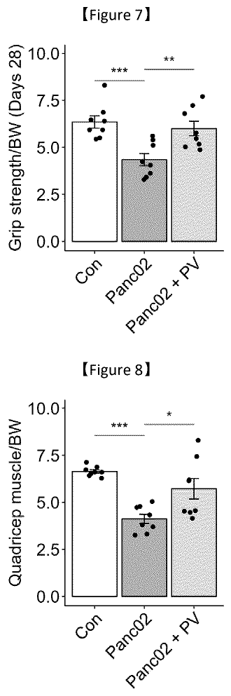

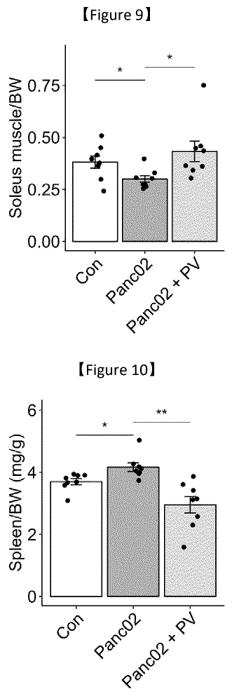

As a result, as shown in FIGS. 8 and 9, compared to the control group, it was confirmed that the Panc02 group (cancer cachexia-induction group) showed a decrease in the weight of quadricep muscle and soleus muscle, and the Panc02+PV group administered P. vulgatus showed a significant increase in the weight of quadricep muscle and soleus muscle.

### [Example 7] Confirmation of Spleen Weight Gain and Loss in Mice Induced with Cancer Cachexia Using Panc02 Pancreatic Adenocarcinoma Cell Line

After a total of 3 weeks of administration of P. vulgatus in [Example 5] above, the spleen weights in all-treatment groups were determined.

As a result, as shown in FIG. 10, compared to the control group, it was confirmed that the Panc02 group (cancer cachexia-induction group) showed an increase in spleen weight, and the Panc02+PV group administered P. vulgatus showed a significant decrease in spleen weight.

### [Example 8] Confirmation of Myofiber Improvement in Mice Induced with Cancer Cachexia Using Panc02 Pancreatic Adenocarcinoma Cell Line

After a total of 3 weeks of administration of P. vulgatus in [Example 5] above, the tibialis muscle of in all-treatment groups was stained with H&E (hematoxylin and eosin) and histological examination was performed.

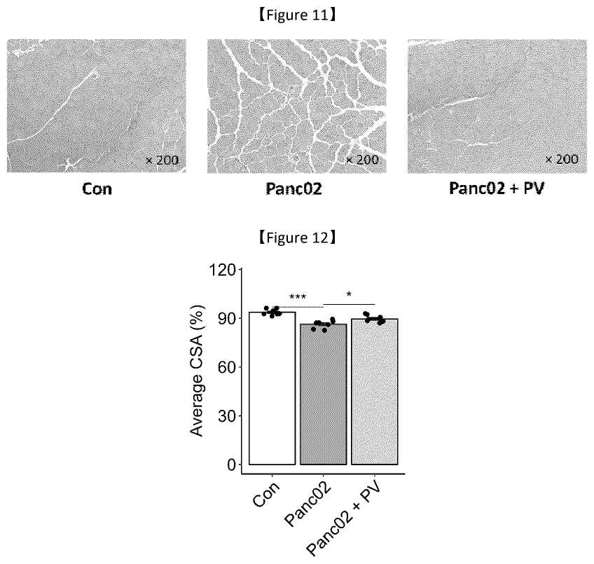

As a result, as shown in FIGS. 11 and 12, it was confirmed that the Panc02+PV group administered P. vulgatus showed a significant increase in muscle fiber (cross-sectional area %) compared to the Panc02 group.

1. A method for increasing muscle mass, improving muscle function, or preventing or treating muscular disease, comprising administering or taking a composition comprising Phocaeicola vulgatus or a culture thereof as an active ingredient to an individual in need.

2. The method of claim 1, wherein the muscle disease is one or more diseases selected from the group consisting of sarcopenia, muscular atrophy, cachexia, myasthenia, muscular dystrophy, myotonia, hypotonia, muscular weakness, muscular dystrophy, atony, amyotrophic lateral sclerosis, and inflammatory myopathy.

3. The method of claim 1, wherein the composition increases muscle strength.

4. The method of claim 1, wherein the composition increases muscle weight.

5. The method of claim 4, wherein the muscle is one or more muscle selected from the group consisting of quadricep muscle, soleus muscle, gastrocnemius muscle and tibialis muscle.

6. The method of claim 1, wherein the composition reduces spleen weight.

7. The method of claim 1, wherein the composition increases muscle fiber.

8. A quasi-drug composition for increasing muscle mass, improving muscle function, or preventing or ameliorating muscular disease, comprising Phocaeicola vulgatus or a culture thereof as an active ingredient.

9. A food composition for increasing muscle mass, improving muscle function, or preventing or ameliorating muscle disease, comprising Phocaeicola vulgatus or a culture thereof as an active ingredient.
